(19) United States
(12) Patent Application Publication
(10) Pub. No.: US 2011/0259128 A1
(43) Pub. Date: Oct. 27, 2011

(54) Integrity Evaluation of Prestressed Concrete Girders

(75) Inventors: Paul H. Ziehl, Irmo, SC (US); Francisco A. Barrios, Santa Marta (CO)

(73) Assignee: University of South Carolina, Columbia, SC (US)

(21) Appl. No.: 13/092,281

(22) Filed: Apr. 22, 2011

Related U.S. Application Data
(60) Provisional application No. 61/326,886, filed on Apr. 22, 2010.

(51) Int. Cl. G01N 33/38

(52) U.S. Cl. 73/866

(57) ABSTRACT

A novel and practical methodology that accounts for the specific mechanical features of a prestressed concrete girder (elastic stiffness, cracking moment, fully cracked inertia, and ultimate capacity) and allows the objective delimitation of its damage levels is presented. Results from this procedure show excellent correlation when compared to the experimentally defined damage thresholds. Also, the global integrity parameter is proposed as a new criterion for damage diagnosis and performance evaluation of prestressed concrete girders within the intermediate and heavy damage zones, showing excellent correlation with the experimental information obtained during testing.

### CROSS-REFERENCE TO RELATED APPLICATION

The present application is based on and claims priority to U.S. Provisional Application Ser. No. 61/326,886 having a filing date of Apr. 22, 2010, which is incorporated by reference herein.

### BACKGROUND

In recent years structural evaluation of existing infrastructure has become a critical subject in civil engineering. Unfortunately the existing load testing methodologies for integrity assessment of civil structures such as the 24 hour load test method (24 h LT) and the cyclic load test method (CLT) have been recently questioned for not providing an accurate diagnosis of the deterioration in the system, and also inducing new damage during the testing procedure.

In light of these circumstances significant efforts have been placed on developing nondestructive techniques such as acoustic emission monitoring (AE) that can effectively assess the integrity of a structure without causing unnecessary deterioration. However, AE methods still face several challenges regarding the subjectivity of their criteria and the lack of quantifiable parameters, which can be directly related to the mechanical response of the system.

Some authors have stated that an integrated approach of the CLT with AE techniques will overcome these difficulties and constitute an effective and true nondestructive evaluation methodology. At this time, various attempts to combine both approaches into a single method have shown promising results yet most of the before mentioned drawbacks still remain unsolved.

As such, a method to effectively assess the integrity of a structure without causing unnecessary deterioration would be desirable. A method that utilizes AE techniques would be particularly beneficial.

### SUMMARY

Aspects and advantages of the disclosure will be set forth in part in the following description, or may be obvious from the description, or may be learned through the practice of the disclosure.

In certain embodiments of the present disclosure, a method for the estimation of damage zones in prestressed girders is described. The method includes selecting a prestressed girder for identification of damage zones and estimating damage zones by taking account cracking moment of the girder, ultimate load of the girder, fully cracked inertia of the girder, and elastic stiffness of the girder.

In still other embodiments of the present disclosure, a method for the estimation of damage zones in prestressed girders is described. The method includes estimating damage zones by using a global integrity parameter (GIP).

These and other features, aspects and advantages of the present disclosure will become better understood with reference to the following description and appended claims.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates damage zones using I_{DL} as a performance descriptor;

FIG. 2 illustrates cracking pattern for I_{DL}=16% (SCLC-2 fatigue girder);

FIG. 3 illustrates cracking pattern for I_{DL}=47% (SCLC-2 fatigue girder);

FIG. 4 illustrates cracking pattern for I_{DL}=65% (SCLC-2 fatigue girder);

FIG. 5 illustrates cracking pattern for I_{DL}=29% (SCC-1);

FIG. 6 illustrates cracking pattern for I_{DL}=65% (SCC-1);

FIG. 7 illustrates GIP values (lightweight girders);

FIG. 8 illustrates GIP values (normal weight girders);

FIG. 9 illustrates UCM values (all girders);

FIG. 10 illustrates CR vs. 1-LR (SCC-1);

FIG. 11 illustrates CR vs. 1-LR (HESC);

FIG. 12 illustrates CR vs. 1-LR (SCC-2 fatigue girder);

FIG. 13 illustrates CR vs. 1-LR (SCLC-2 fatigue girder);

FIG. 14 illustrates damage thresholds from AE on the SIL loops (SCC-1);

FIG. 15 illustrates damage thresholds from AE on the SIL loops (HESC);

FIG. 16 illustrates damage thresholds from AE (SCC-2 fatigue girder);

FIG. 17 illustrates damage thresholds from the AE (SCLC-2 fatigue girder);

FIG. 18 illustrates definition of the arch of damage on the CR vs. LR plots;

FIG. 19 illustrates linear distance of each loadset to the point of no-damage (normal weight girders);

FIG. 20 illustrates angular distance of each loadset to the point of no-damage (normal weight girders);

FIG. 21 illustrates arch of damage of each loadset (normal weight girders);

FIG. 22 illustrates linear distance of each loadset to the point of no-damage (lightweight girders);

FIG. 23 illustrates angular distance of each loadset to the point of no-damage (lightweight girders);

FIG. 24 illustrates arch of damage of each loadset (SCLC);

FIG. 25 illustrates I_{G} results with AE for lightweight girders;

FIG. 26 illustrates I_{G} results with AE for normal weight girders;

FIG. 27 illustrates GIP with AE for lightweight girders (P_{T}=128 kips);

FIG. 28 illustrates GIP with AE for normal weight girders (P_{T}=96 kips);

FIG. 29 illustrates GIP with AE for lightweight girders (P_{T}=160 kips);

FIG. 30 illustrates GIP with AE for SCC girders (P_{T}=128 kips);

FIG. 31 illustrates load vs. Displacement from the FE model (lightweight girders);

FIG. 32 illustrates load vs. Displacement from M-C analysis (normal weight girders);

FIG. 33 illustrates theoretical damage thresholds (SCLC girders);

FIG. 34 illustrates theoretical damage thresholds (normal weight girders);

FIG. 35 illustrates specimen details;

FIG. 36 illustrates load vs. Displacement (STD-M-A);

FIG. 37 illustrates load vs. Displacement (STD-M-B); and

FIG. 38 illustrates load vs. Displacement (STD-M-D).

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure describes the capability of the cyclic load test method (CLT) as well as acoustic emission (AE) evaluation to identify damage and assess structural integrity when applied to prestressed self-consolidating lightweight concrete (SCLC) and self-consolidating concrete (SCC) girders, and further describes a new methodology for the objective identification of the damage levels present in a tested member for structural evaluation. Further, a combination of the CLT procedure and AE criteria into one methodology that can provide a better assessment of the structural integrity in prestressed flexural members is disclosed.

The development of reliable, economic, and practical load testing methods as well as other nondestructive evaluation procedures is a priority in structural engineering. In lack of these methodologies, modern construction techniques, new materials, and novel analysis methods cannot be implemented in a safe manner, as well as outmoded and degraded structures cannot be guaranteed to work properly. The present disclosure enhances current capabilities in static structural evaluation and health monitoring through the improvement of the CLT method and AE techniques.

In order to obtain a meaningful frame for integrity assessment, the damage zones must be clearly delimited and unambiguously defined. In order to do this, the deviation from linearity index (I_{DL}) can be used as a tool for delimiting the damage levels.

Based upon data gathered from CLT testing of SCLC girders, the thresholds for defining the damage level zones have been defined as follows: minor damage for I_{DL}<15%, moderate or intermediate for 15%<I_{DL}<35% and heavy or severe damage for I_{DL}>35%. These limits are also adequate to delimitate the damage zones observed during the testing of an identical set of three prestressed girders (one high-early-strength concrete (HESC) and two SCC specimens) with different deck geometry.

It is important to mention that I_{DL} values are sensitive to the failure mode, and therefore the ranges recommended in the present disclosure are applicable only to prestressed concrete beams with a flexure failure mode. Nonetheless, an I_{DL}=25% was initially formulated and successfully implemented as an indication of heavy damage in reinforced concrete beams with a failure mode in flexure.

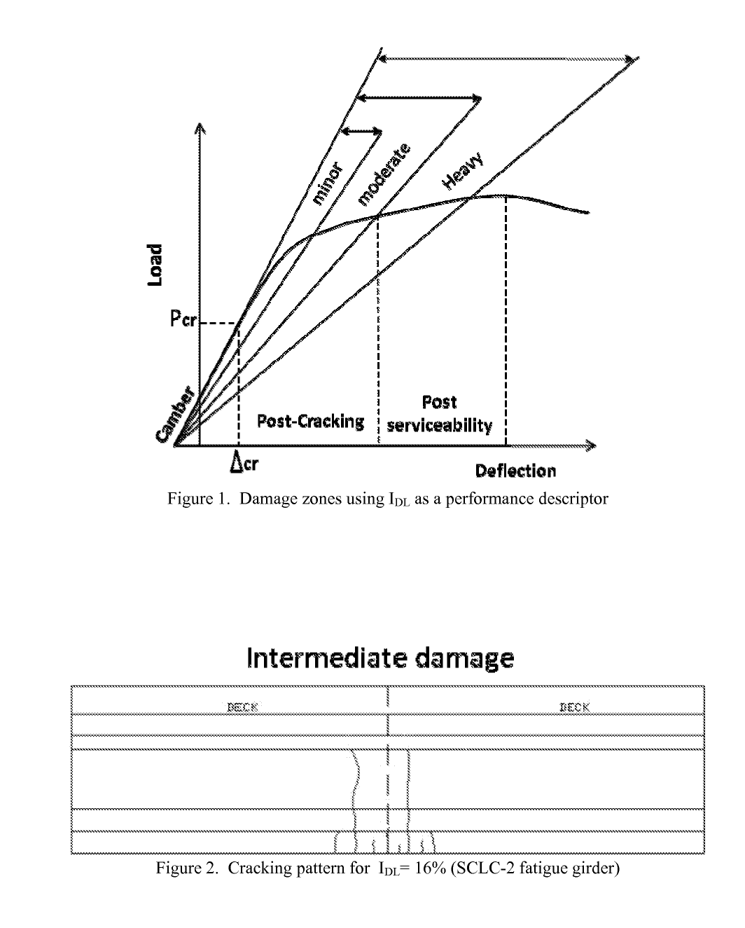

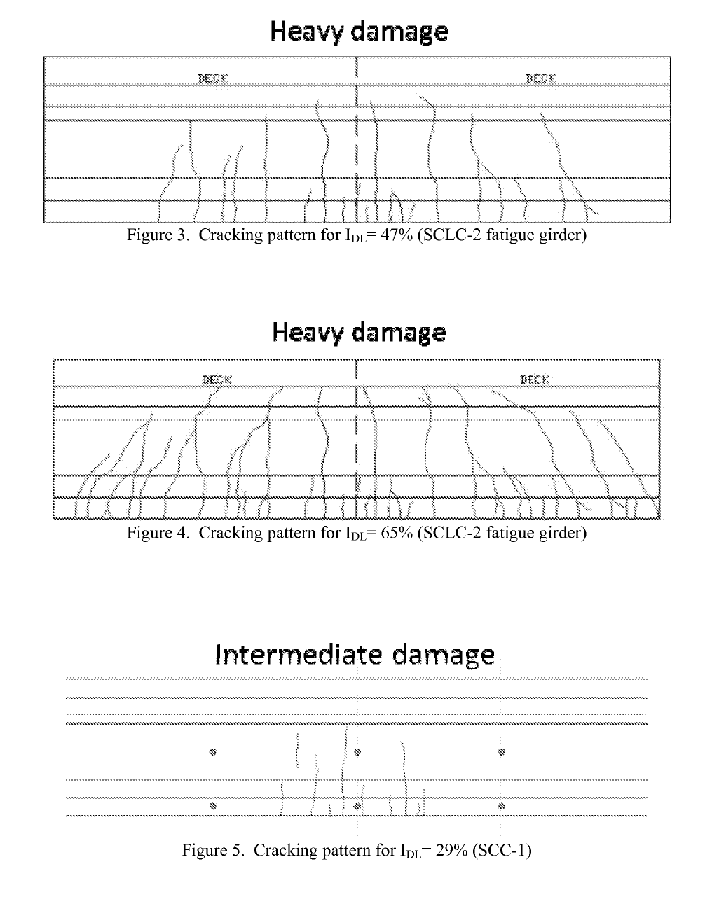

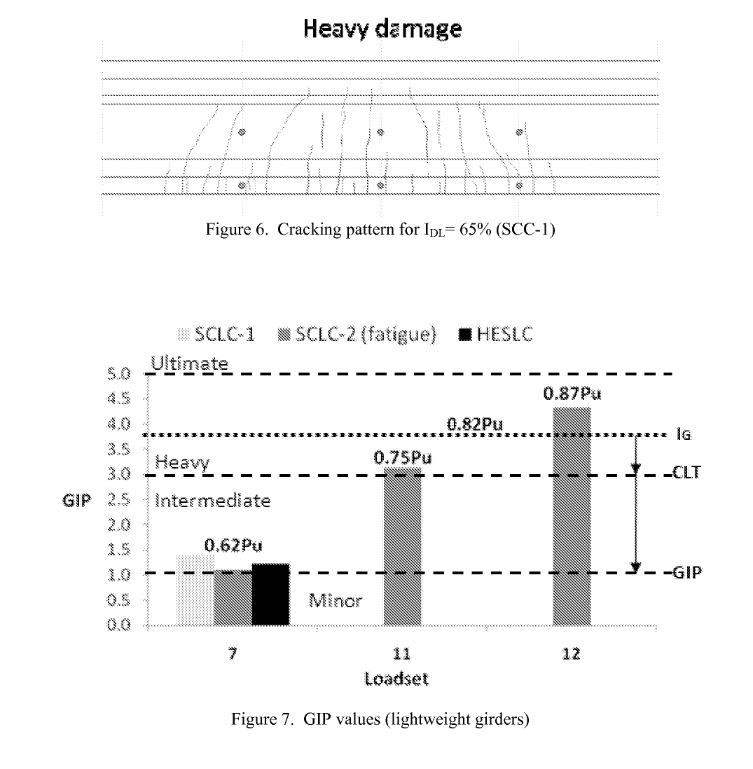

In order to reassure the viability of the I_{DL} as a damage descriptor for the minor and intermediate damage zones, cracking patterns corresponding to the CLT testing of the SCLC and SCC girders with their respective I_{DL} values are shown in FIGS. 1-6.

The I_{DL} criterion can be a powerful tool for monitoring the response of prestressed flexural members during in situ load testing, regardless of the type of concrete used in their manufacture. However, the criterion must be used with care. Unexpected sources of nonlinearity along with combination of failure modes can bias the evaluation providing inadequate safety margins. Also, at this moment there is no closed form solution or methodology that can provide a more precise delimitation of the damage zones using the specific structural features (such as cross sectional area, modulus of elasticity, prestress ratio, or the like) of a given member. Additionally, previous evidence indicates that a member that has been subjected to prior damage may exhibit rather low I_{DL} values under certain circumstances. It appears that this situation may occur in flexural members with a low prestress ratio and/or low amount of longitudinal reinforcing, which usually exhibit a cracking moment rather close to the nominal capacity of the member. In these cases the working range of the I_{DL} becomes significantly narrow and additional criteria become indispensable to prevent the exertion of damage or even the collapse of the system during load testing.

To assure an unbiased and accurate evaluation of the safety and serviceability of civil structures, linguistic definitions of the damage zones must be accompanied by a quantitative criterion that can reliably identify when a member is functioning at a particular level of integrity.

Also the criterion must take into account the specific mechanical properties of the member to be tested, in order to assure the accuracy and the safety of the test. In accordance with the present disclosure, a new method that takes into consideration the cracked inertia of the member, its ultimate load, fully cracked inertia and its elastic stiffness is proposed with this purpose, see FIG. 31.

This approach aims to represent the three damage levels as percentages of the total deviation from linearity at ultimate I_{DLU}. To do this the fully cracked inertia (Icr) is defined as the line from the cracking load (or cracking moment) to the ultimate capacity, and the effective inertia (Ie) as the secant from the origin to the same point. The cracking moment of a simply supported fully prestressed member with constant tendon eccentricity can be computed as:

$${M}_{Cr} = {S}_{b} [ \frac{{P}_{e}}{{A}_{c}} ( 1 + \frac{e \times {C}_{b}}{{r}^{2}} ) + 7.5 λ \sqrt{{f}_{c}^{′}} ] \qquad ( 1 )$$

And the fully cracked inertia of a fully prestressed member as

$${I}_{Cr} = {n}_{p} {A}_{p s} {d}_{p}^{2} ( 1 - 1.6 \sqrt{{n}_{p} \times {ρ}_{p}} ) \qquad ( 2 )$$

Where, Ig is the gross moment of inertia, Sb is the modulus of the composite section at the bottom fibers, Cb is the distance from the center of gravity of the girder section to the extreme tension fibers, Pe is the effective prestress force, Ac is the gross sectional area of the girder, e is the eccentricity of the tendons from the girder section center of gravity, r is the radius of gyration of the girder, Mu is the ultimate moment, nₚ is the young modulus ratio, Aₚₛ is the area of prestressing steel, dₚ is the distance from the top of the section to the centroid of prestress, ρₚ is the prestress reinforcing ratio, and λ is equal to 1.0 for normal weight and 0.75 for lightweight concrete. From here, the effective inertia can be computed graphically and the deviation from linearity at ultimate can be calculated as:

$${I}_{DLU} = ( 1 - \frac{{I}_{e}}{{I}_{o}} ) \times {10}_{0} \qquad ( 3 )$$

Next, the three damage zones can be estimated as follows,

I_{DL-MINOR}≦0.2×I_{DLU} (4)

0.2×I_{DLU}<I_{DL-INTERMEDIATE}≦0.45×I_{DLU} (5)

0.45×I_{DLU}<I_{DL-HEAVY} (6)

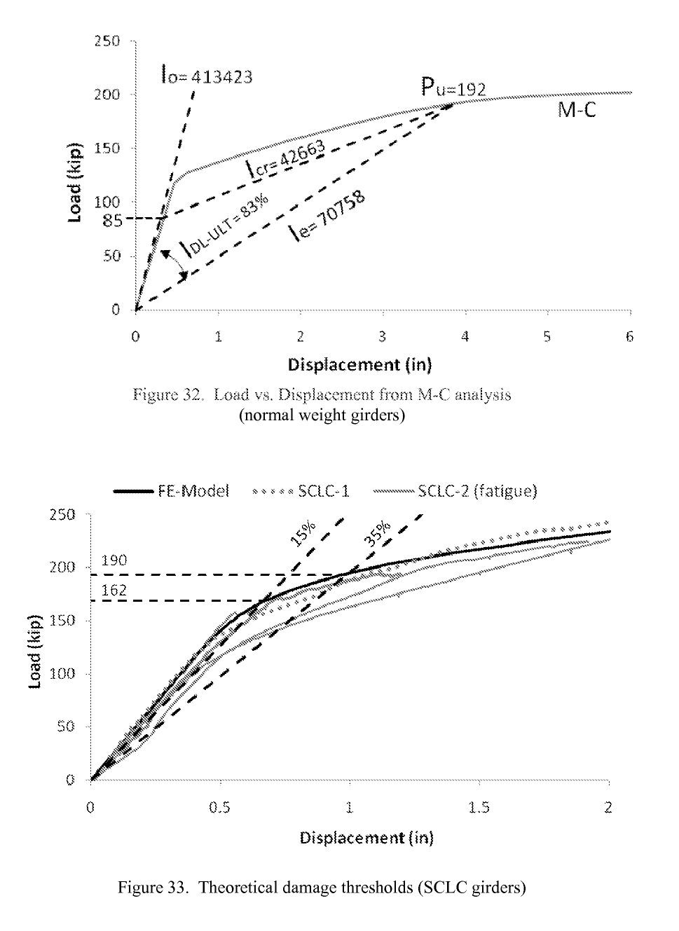

The theoretical damage thresholds presented in Table 1.1, are calculated based on the I_{DLU} from the FE results for the SCLC girders (FIG. 31), and on the I_{DLU} from the moment-curvature (M-C) for the SCC beams (FIG. 32), since no FE model was built for these specimens. The M-C analysis of the SCC girders was carried out using the program RESPONSE 2000 developed at the University of Toronto, which performs sectional analysis of reinforced and prestressed concrete members based on the modified compression field theory.

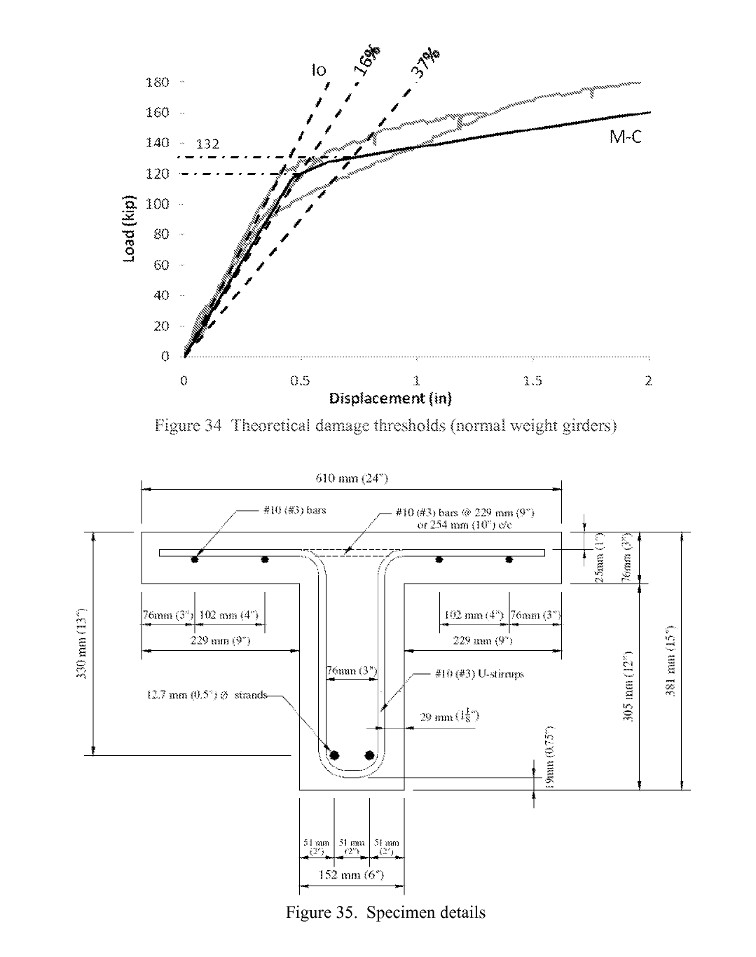

Theoretical damage thresholds are illustrated in FIGS. 33 and 34 for SCLC and SCC girders.

TABLE 1.1
I_{DLU} for SCC and SCLC girders

| | | Theoretical (%) | | Experimental (%) | |
|---|---|---|---|---|---|
| Girders | I_{DLU} (%) | Minor-Int | Int-Heavy | Minor-Int | Int-Heavy |
| SCC | 83 | 17 | 37 | 15 | 35 |
| SCLC | 78 | 16 | 35 | | |

From Table 1.1, it is observed that the theoretical values of I_{DLU} and their corresponding damage thresholds show very good agreement for all giders (SCLC and SCC).

To further explore the consistency of the methodology, deterioration levels were determined for three prestressed beams. The beams were tested and evaluated with the current CLT criteria; therefore the minor and intermediate zones of worsening were not established, and the heavy damage level was assumed to start at an I_{DL} equal to 25%.

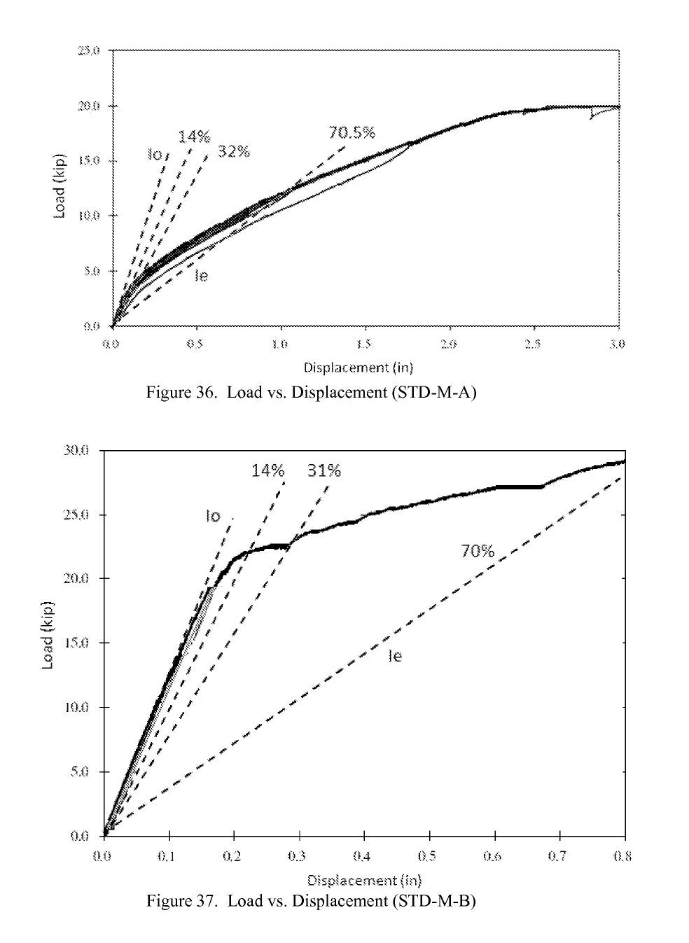

Details of the specimens are shown in FIG. 35 and Table 1.2. The results from the analysis are summarized in Table 1.3 and presented in FIGS. 36, 37 and 38.

TABLE 1.2
Specimen Details

| Beam | Span (in) | Pcr (Kip) | Pu (kip) | f′c (psi) | Io (in⁴) | Icr (in⁴) | Ie (in⁴) | IDLU (%) |
|---|---|---|---|---|---|---|---|---|
| STD-M-A | 276 | 11.5 | 14.11 | 6,320 | 3,750 | 270 | 1,106.94 | 70.5 |
| STD-M-B | 196 | 18.9 | 23.035 | | | | 1,130.48 | 69.9 |
| STD-M-D | 116 | 44.2 | 52.02 | | | | 2,174.45 | 42.0 |

The proposed methodology offers important advantages when assessing the integrity of a prestressed flexure member. First, it allows the location of the member within the minor and intermediate damage zones, using thresholds that are specific to the mechanical properties of the girder. In doing so, the method also permits the minimization of the damage during testing, since the loading procedure can be stopped before reaching undesired levels of damage in the member.

TABLE 1.3
CLT results with new criterion

| Beam | Cycle | 3A | 4A | 7A | 8A | 9A | 10A |
|---|---|---|---|---|---|---|---|
| STD-MA | IDL (%) | 8.4 | 8.4 | 26.3 | 31.3 | 44.4 | 44.6 |
| | Damage | Minor | Minor | Intermediate | Intermediate | Heavy | Heavy |
| STD-M-B | IDL (%) | 4.2 | 4.5 | 28.8 | 31.1 | 64 | 65.2 |
| | Damage | Minor | Minor | Intermediate | Heavy | Heavy | Heavy |
| STD-M-D | IDL (%) | 1.7 | 8.3 | — | — | — | — |
| | Damage | Minor | Minor | — | — | — | — |

FIG. 36 shows the damage thresholds found for specimen STD-M-A. This specimen had been pre-cracked and taken close to yielding before the application of the CLT procedure. The figure shows how the beam rapidly diverges from its elastic stiffness at load levels below 30% of its calculated ultimate capacity (14.1 kips).

In a real load test the theoretical damage thresholds proposed here, would identify this rapid transition of the member from minor to severe worsening as a clear indication of poor structural integrity impeding the exertion of further damage or even the collapse of the system.

These results are in opposition to the idea of using a cracked inertia as the reference line for the evaluation of the I_{DL}. This practice has been used in conjunction with the concept that initial cracking in a beam can generate a high deviation from linearity without a reduction of the ultimate capacity of the girder. This thought is reasonable under the current CLT procedure, since its criteria were designed only to indicate the presence of severe damage, and therefore nonlinear behavior that did not result in any quantifiable worsening of the structure could not be classified as such.

In fact, the use of a cracked inertia as a reference line on beam STD-M-A will result in extremely low I_{DL} values, and thus the severe damage in the member will not be acknowledged.

FIG. 37 shows the calculated thresholds for the STD-M-B specimen. This beam was undamaged before the CLT, and the graphs show a good correlation between the theoretical limits and the load vs. displacement behavior of the girder.

At first sight, delimitation of the zones might appear too conservative, however it is good to remember that minor and intermediate regions do not allow the presence of significant plastic deformations and that the member should be entirely repairable. Also, the main objective of the methodology is to assess the integrity of the system minimizing the amount of new damage during testing.

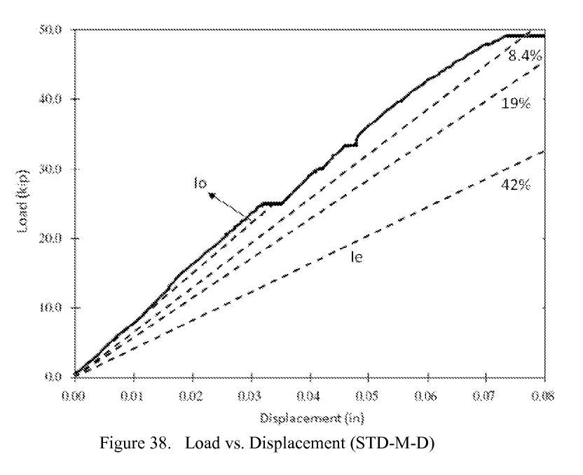

FIG. 38 presents results from specimen STD-M-D. This specimen was arranged to fail in a shear mode by considerably shortening its span to 116 in. It is observed that the specimen showed a strong linear behavior up to a load level close to ultimate impeding the detection of damage in the girder.

These results pose an important problem for the integrity assessment using the deviation from linearity index. First, mid-span displacement is greatly reduced in members with a failure mode in shear, and thus they will generally experience very low values of the I_{DL} for dangerously high levels of load. This drawback might be partially solved by using an I_{DL} based on strain rather than displacement, but since the nonlinear behavior in shear is controlled by different mechanisms from flexure, further investigation is needed on the subject. Another possibility will be the inclusion of different damage parameters (such as AE criteria) that are able to account for damage without the presence of nonlinearity.

The present disclosure also presents the structural evaluation of specimens with a new approach using the parameter of deviation from linearity to overcome difficulties highlighted in the current methodologies. The global integrity parameter (GIP) is described herein as an alternative that significantly improves the integrity assessment over its counterpart the global performance index I_{G}, and the current I_{DL} criterion in the CLT.

The GIP uses a new methodology for the identification of the damage zones in a flexural member, to improve the assessment of the integrity while minimizing the amount of damage caused to the structure during the loading procedure. This new methodology relies on the deviation from linearity index and removes all the other indices used in the I_{G} since they were either unreliable or had a very restricted range of application.

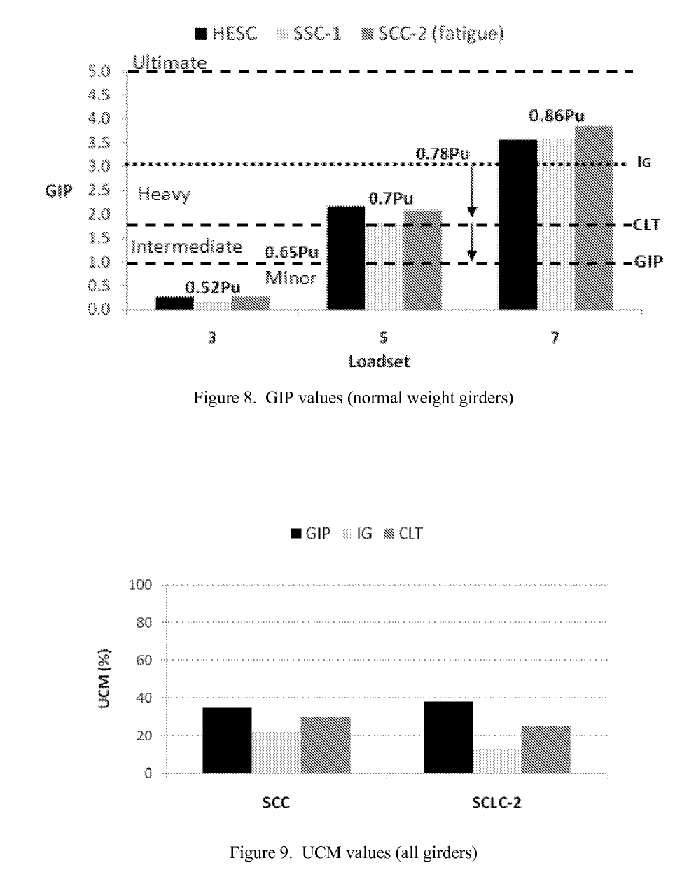

The GIP targets the maximum test load as corresponding to the minor-intermediate damage threshold, and compares values of the deviation from linearity obtained during testing to the corresponding damage threshold. The GIP is defined as:

$$G I P = ( \frac{{I}_{DL}}{0.2 {I}_{DLU}} ) \leq 1.0 \qquad ( 7 )$$

Where I_{DLU} is the theoretical deviation from linearity at ultimate and the I_{DL} is the experimental deviation from linearity experienced by the member at any load level during testing. Computed GIP values are shown in FIGS. 7 and 8, as well as summarized in Table 2.1 and 2.2 for SCLC and SCC girders respectively.

TABLE 2.1
GIP values with no dummy loadsets (lightweight girders)

| | Loadset | 5 | 7 | 11 | 12 | I_{DLU} |
|---|---|---|---|---|---|---|
| Gider | % of Pu | 0 | 0 | 0 | 0 | (%) |
| HESLC | I_{DL} (%) | 0.0 | 18 | — | — | 78 |
| | GIP | 0.0 | 1.2 | — | — | |
| SCLC-1 | I_{DL} (%) | 0.0 | 21 | — | — | |
| | GIP | 0.0 | 1.4 | — | — | |
| SCLC-2 | I_{DL} (%) | 0.0 | 16 | 47 | 65 | |
| | GIP | 0.0 | 1.1 | 3.1 | 4.3 | |

I_{DLU}: deviation from linearity at ultimate

From FIGS. 7 and 8, it can be observed that the GIP offers a higher sensitivity for damage detection than both the I_{G} and the current I_{DL} of the CLT criteria. In the case of the SCLC girders, the GIP reduces the level of load at which the criterion reaches unity to 62% of the theoretical ultimate capacity of the member. This constitutes an important reduction in the applied load when compared to the 87% required by the I_{G} and to the 75% by the CLT. In addition the GIP provides the theoretical locations of the damage levels (dashed lines), so the amount of damage indicated by every loadset can be estimated more accurately.

In the case of the SCC girders, the GIP reduces the load at which the criterion is failed to 65% of ultimate, outperforming the values of 70% and 78% given by the CLT and the I_{G} respectively (see FIG. 8). Values of the corresponding ultimate capacity margins (UCM) for SCLC and SCC girders are plotted in FIG. 9.

TABLE 2.2
GIP values no dummy loadsets (normal weight girders)

| | | Loadset | | | |
|---|---|---|---|---|---|
| | | 3 | 5 | 7 | I_{DLU} |
| Gider | % of Pu | 52 | 69 | 86 | (%) |
| HESC | I_{DL} (%) | 4 | 35 | 57 | 83 |
| | GIP | 0.3 | 2.2 | 3.6 | |
| SCC-1 | I_{DL} (%) | 3 | 29 | 57 | |
| | GIP | 0.2 | 1.8 | 3.6 | |
| SCC-2 | I_{DL} (%) | 4 | 33 | 62 | |
| | GIP | 0.3 | 2.1 | 3.9 | |

It is evident that the GIP index possesses a higher capability for damage detection at lower levels of load when compared to the current available criteria from the CLT and the global performance index (I_{G}). In addition, values of the GIP can be directly related to the damage levels in a member, offering a consistent and meaningful integrity assessment.

As described herein, a numerical model of the girders is illustrated and the results from the analysis are used to confirm the advantages of the GIP criterion for integrity assessment of prestressed flexural members.

The present disclosure also provides a new approach for the processing and interpretation of the calm ratio (CR) vs. load ration (LR) data as presented below. The calm ratio describes the AE activity during the unloading part of the cycles. Also known as felicity ratio or concrete beam integrity (CBI), the load ratio (LR) it is a critical parameter for AE monitoring.

The new methodology includes the index of deviation from linearity I_{DL} as an external parameter to calibrate the CR vs. LR analysis. In doing so, this combined approach enjoys the advantages of both criteria, using the objective delimitation of the damage zones proposed here with the I_{DL}, and putting it together with the higher sensitivity of the AE monitoring and its capability of damage sensing regardless of linear behavior.

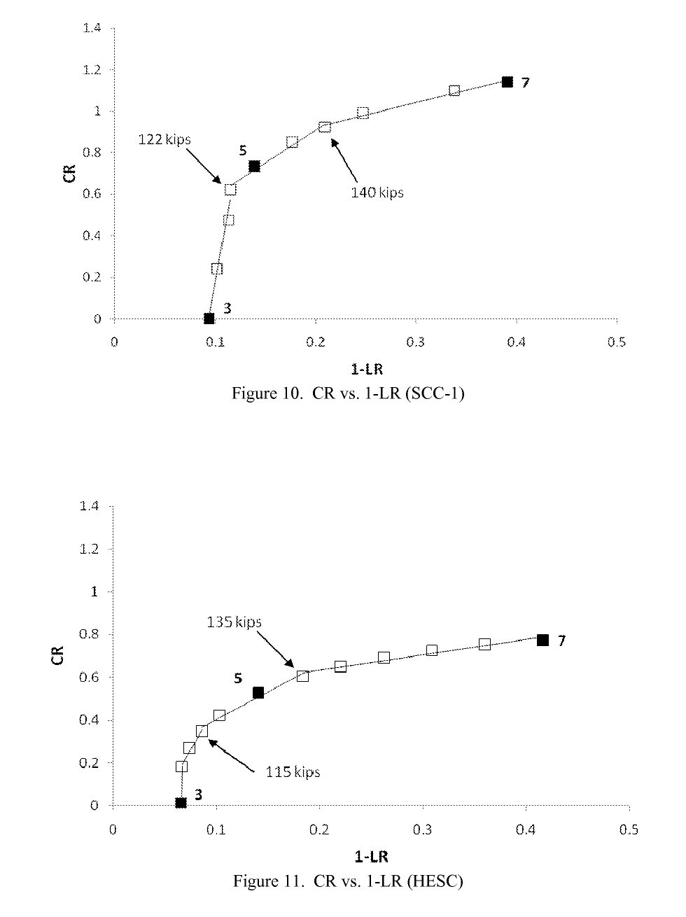

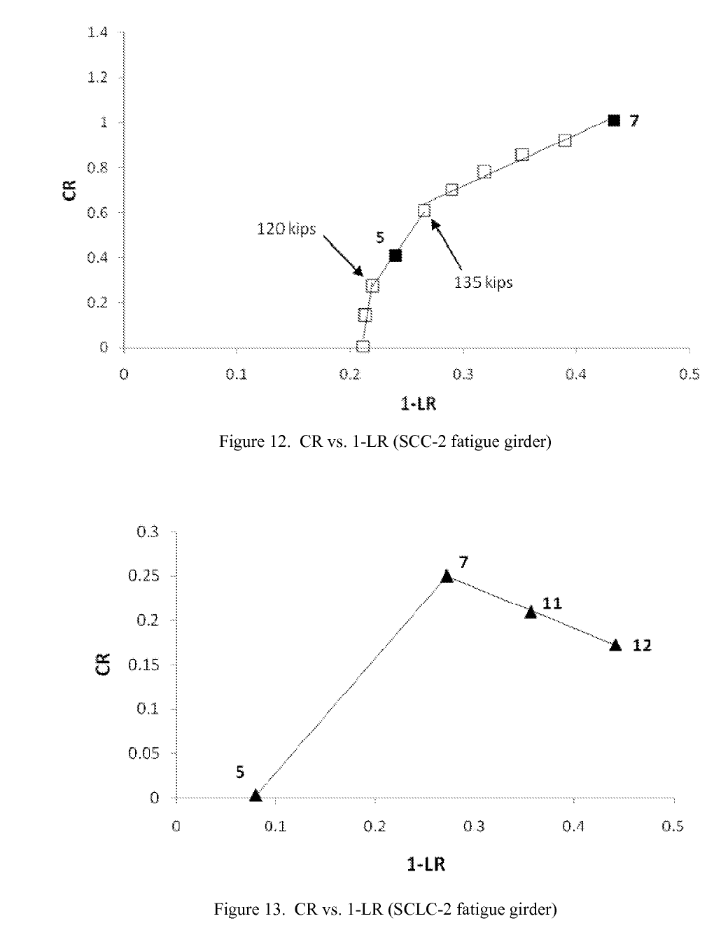

Plots of the CR vs. 1-LR criterion obtained following all recommendations described previously are shown on FIGS. 10 through 11, load values at the points where slop changes are shown for posterior comparison with the structural integrity loops. Again, hollow squares represent values that were interpolated and extrapolated using the curves obtained for CR and LR. The LR criterion was replaced by 1-LR only for convenience, so the curves will increase rightward and upward.

It is evident that there is a common pattern in all three beams that describes the accumulation of damage during loading. These figures represent a significant improvement in clarity when compared to the plots presented in all of the previous investigations where no clear trend could be identified.

Also the curves plotted for the normal weight girders can be easily divided into three segments; these sections indicate that the CR criterion is more sensitive within very early stages of damage, while the LR is predominant in later phases of deterioration where CR values remain relatively constant. This continues up to a point where cracks do not close anymore due to the large accumulation of plastic deformation in the member, and the CR values start to decrease rapidly (loadsets 11 and 12 for SCLC-2 shown in FIG. 13).

At this point, the typical assumption is that each change in slope in the CR vs. LR graph corresponds to the transition between the damage zones previously portrayed. However, since currently AE activity does not have a quantitative description, this is still conjecture. Moreover, in order to successfully merge AE evaluation with the I_{DL} criterion, a coherent and equivalent description of the damage process given by both parameters must be obtained.

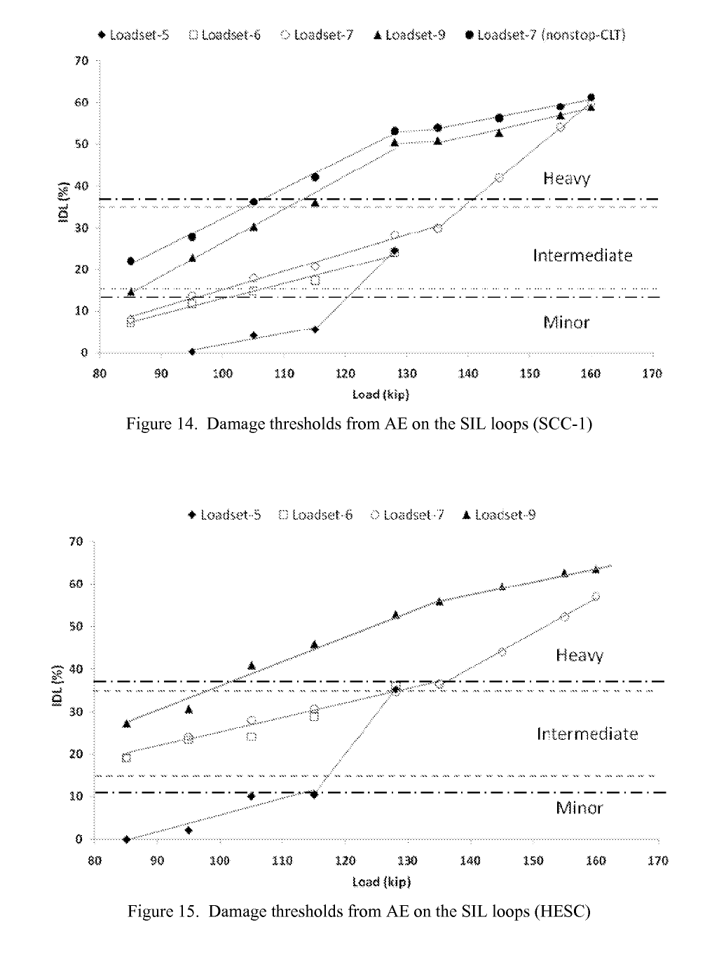

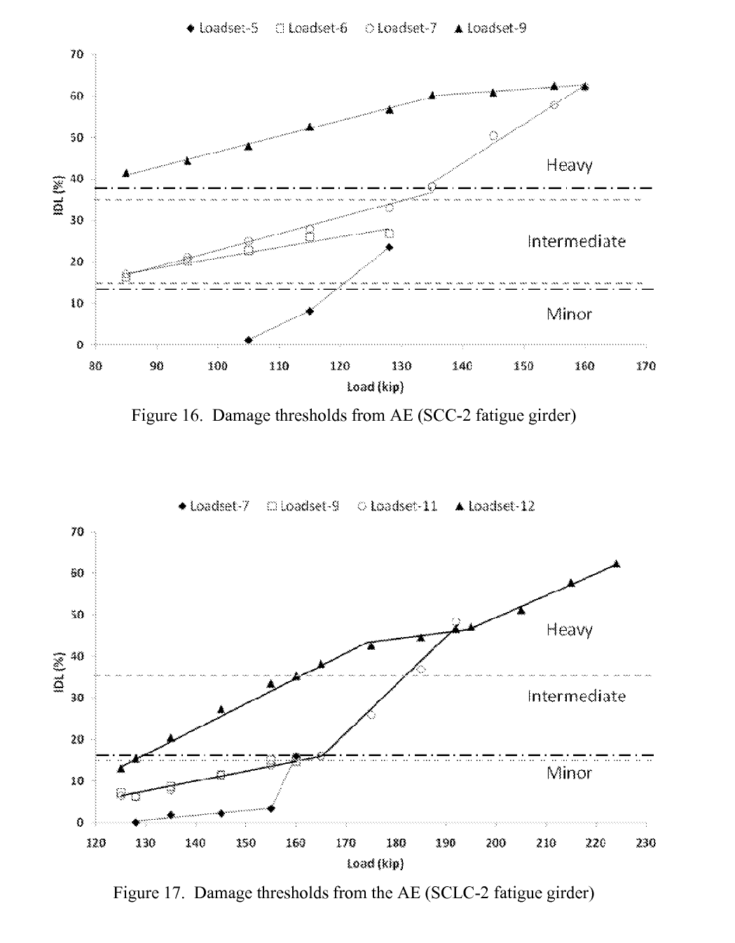

For this purpose, load values at the transition points on the CR vs. LR plots are located on the structural integrity loops to estimate the corresponding I_{DL} values, see FIGS. 14 through 16 (bold dash-dot lines).

It is very clear that the changes in slope on the CR vs. LR represent physical changes in the member that match up the worsening phenomena described by the I_{DL}. This equivalence constitutes an important advance towards the transformation of the CR vs. LR from a qualitative criterion to a parameter that is quantifiable in terms of damage. For SCLC-2 only the minor-intermediate boundary could be located from the information gathered in the CR vs. LR plot. It seems the SCLC-2 girder jumped from the minor zone directly to the heavy damage level (see FIG. 17).

Here, it is important to remember that the AE monitoring of the present disclosure is mainly focused on cracking. This will generally allow an earlier detection of damage in flexural members. However, due to the sensor layout used here it can overlook other deterioration phenomena that may occur simultaneously such as concrete plastic deformation. This might result in the narrowing or even disappearance of an entire damage region and hence reinforces the importance of combining nonlinearity with AE activity for a more effective methodology, as well as the necessity of developing AE techniques for monitoring regions that are dominated by shear or compression.

In addition, it is possible to find more than three changes in slope in a CR vs. LR plot. Without the aid of the I_{DL} it will be harder to discriminate them according to our initial damage level description.

From the results presented above, it can be concluded that the plots of CR vs. LR, when drawn accordingly to the recommendations previously given, can offer very valuable information that can be directly related to the performance of a prestressed flexural member when subjected to the CLT procedure.

However, AE damage descriptor parameters can be proposed in order to provide a more objective assessment of the specimen when tested. Since distance from any loadset to the point of no damage has been previously used with relative success, this parameter will remain as one of the damage descriptors for the CR vs. LR criterion. However, two main changes are worth mentioning, first there is no need to normalize CR values, since the elimination of the dummy loadsets and the use of energy instead of number of hits produce CR values similar in magnitude to the LR values. Also the point of no damage is considered as the position where the first slope of the CR vs. LR curve crosses the horizontal axis, instead of the arbitrary (1,0) (or (0,0) if 1-LR is used).

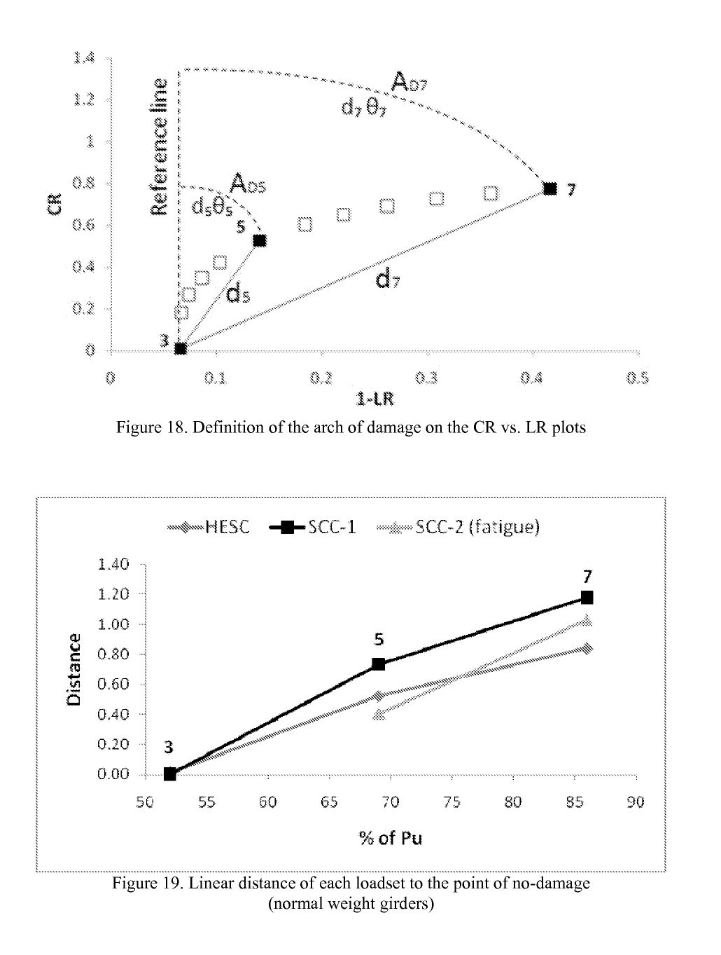

The second AE damage descriptor proposed will be the angular distance (measured in radians) from a no-damage reference vertical line, located at the point of no-damage previously defined. Once both parameters have been computed the arch of damage (A_{D}) can be calculated as the multiplication of both parameters, see FIG. 18.

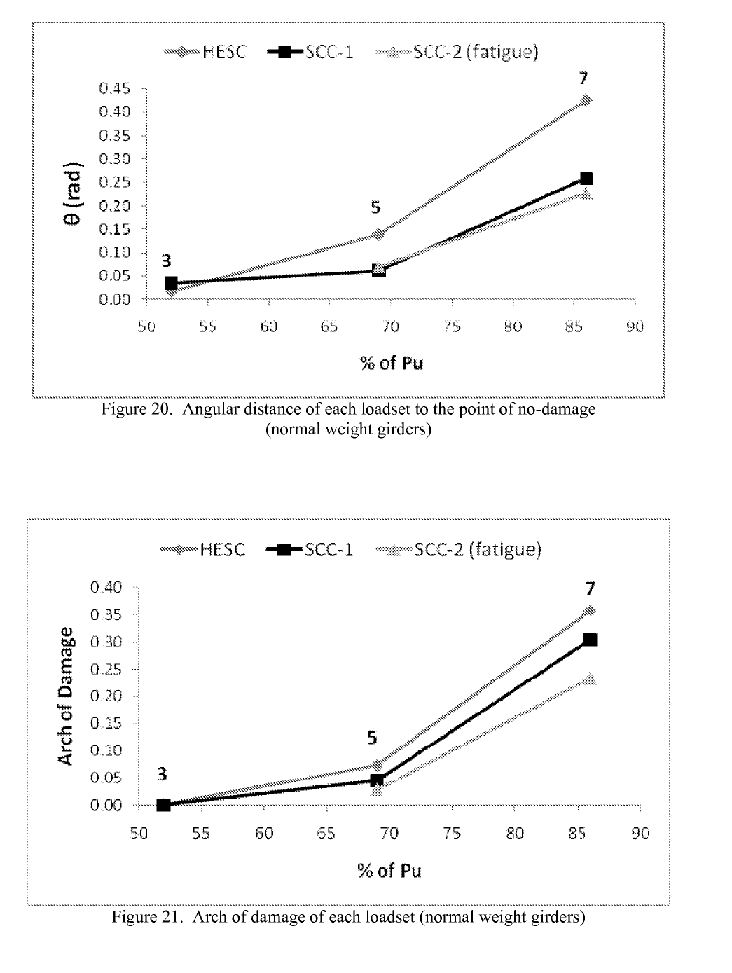

Values of the linear (dᵢ) and angular distance (θᵢ) along with the arch of damage are presented in FIGS. 19 through 21 and summarized in Tables 3 and 4 for all specimens.

TABLE 3
θ, d, and A_{D} (normal weight girders)

Loadset

| | | 3 | 5 | 7 |
|---|---|---|---|---|
| Girder | % of Pu | 52 | 69 | 86 |
| HESC | θ (rad) | 0.02 | 0.14 | 0.4 |
| | Distance | 0.01 | 0.52 | 0.8 |
| | A_{D} | 0.0002 | 0.07 | 0.4 |
| SCC-1 | θ (rad) | 0.03 | 0.06 | 0.3 |
| | Distance | 0.003 | 0.73 | 1.2 |
| | A_{D} | 0.0001 | 0.04 | 0.3 |
| SCC-2 | θ (rad) | 0.03 | 0.07 | 0.2 |
| | Distance | 0.01 | 0.41 | 1.0 |
| | A_{D} | 0.0002 | 0.03 | 0.2 |

For the normal weight specimens, it can be observed in the graphs that, the A_{D} parameter offer a smoother and more consistent damage representation in all of the girders than the linear and the angular distance independently. Also, the linear distance parameter grows faster within the minor damage zone (between loadsets 3 and 5) while the angular distance is more sensitive to damage in the intermediate and the heavy damage levels.

In the case of the lightweight girders, the point of no damage could not be calculated precisely since only loadset 5 was located within the minor damage zone, and therefore the initial slope of the CR vs. LR curve inside this damage region could not be determined. However taken into account the very low amount of damage observed at loadset 5 in all specimens, the point of no damage will be assumed to be located on a vertical line directly below this loadset. Hence, the A_{D} for loadset 5 will be defined in function of the distance to the no damage point only, and the minor damage region will not appear in the angular distance (θ) plot (see FIGS. 19, 20 and 21).

In FIG. 22, deterioration within the minor damage region increases rapidly up to the theoretical minor-intermediate threshold (right at loadset 7), where the linear distance parameter remains almost stable inside a narrow band between 0.30 and 0.35 for a wide range of the load value (62% to 87% of ultimate capacity), this supports the fact that this parameter is more effective inside the minor damage zone.

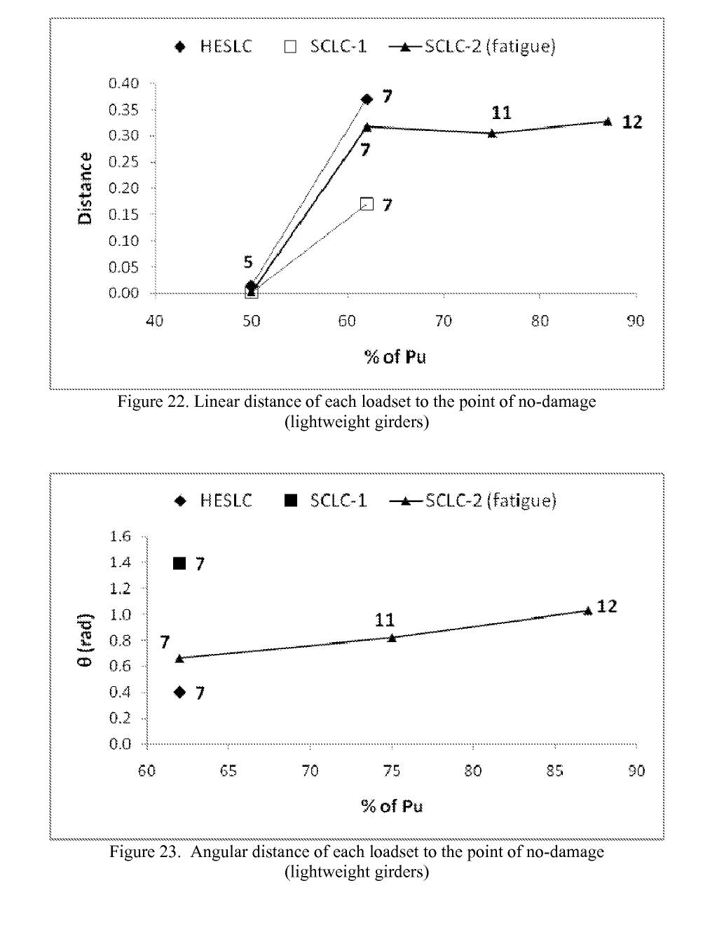

On the other hand, the angular distance parameter is more sensitive to damage within the intermediate and heavy zones and hence increases steadily after loadset 7 following a closely linear pattern (similar to the I_{DL}) for the same load range (see FIG. 23).

It is also worth mentioning that the scatter observed for loadset 7 in both the linear and the angular distance is greatly reduced with the use of the arch of damage. (A_{D}), see FIG. 24.

These observations support the combination of the linear and the angular distance into the A_{D} for an improved integrity assessment when using the CR vs. LR plots.

TABLE 4
θ, d, and A_{D} (lightweight girders)

Loadset

| | | 5 | 7 | 11 | 12 |
|---|---|---|---|---|---|
| Girder | % of Pu | 50 | 62 | 75 | 87 |
| HESLC | θ (rad) | — | 0.4 | — | — |
| | distance | 0.014 | 0.4 | — | — |
| | Arch | 0.014 | 0.1 | — | — |
| SCLC-1 | θ (rad) | — | 1.4 | — | — |
| | distance | 0.001 | 0.2 | — | — |
| | Arch | 0.001 | 0.2 | — | — |
| SCLC-2 | θ (rad) | — | 0.7 | 0.8 | 1.0 |
| | distance | 0.003 | 0.3 | 0.3 | 0.3 |
| | Arch | 0.003 | 0.2 | 0.3 | 0.3 |

A more complete version of the I_{G}, which combines results from the CLT and the AE monitoring in an attempt to get a better assessment of the damage present in a member, is defined as:

$$\begin{matrix}{I}_{G} = \frac{1}{4} [ {α}_{r} {i}_{r} + {α}_{p} \frac{{I}_{r}}{10} + {α}_{DL} \frac{{I}_{DL}}{25} + {α}_{CRLR} \frac{{I}_{CRLR }}{0.45} ] {K}_{G} \leq 1.0 \\ And , & ( 8 ) \\ {I}_{r} = \{ \begin{matrix}2 - {I}_{R} / 95 {I}_{R} \leq 95 \\ 0.2 \vert {I}_{R} - 100 \vert 95 \leq {I}_{R} \leq 105 \\ {I}_{R} / 105 {I}_{R} \geq 105 \end{matrix} & ( 9 )\end{matrix}$$

Where the I_{R}, Ip, I_{DL}, I_{CRLR} are the repeatability, permanency, deviation from linearity and calm vs. load ratio indices, αr, αp, αcrlr, are variables to account for the importance of each index and K_{G} is a multiplier that accounts for the knowledge (load history, previous load tests, reinforcing configuration, and the like) of the structure by the evaluator, and the number of members being tested compared to the total number of similar members in the system.

The permanency and repeatability indices are not included in the computation of the I_{G} along with the AE data since they have shown insensitivity to damage and also questionable reliability. With this modification the evaluation of the I_{G} will be equivalent to the I_{DL} from the CLT method, therefore, the values from the latter will be used for comparison and illustration of the effect that AE criteria produce in the computation of the structural performance indices. Also, the cumulative signal strength ratio parameter (I_{CSSR}) is excluded from the analysis since it did not provide good correlation with damage as previously discussed.

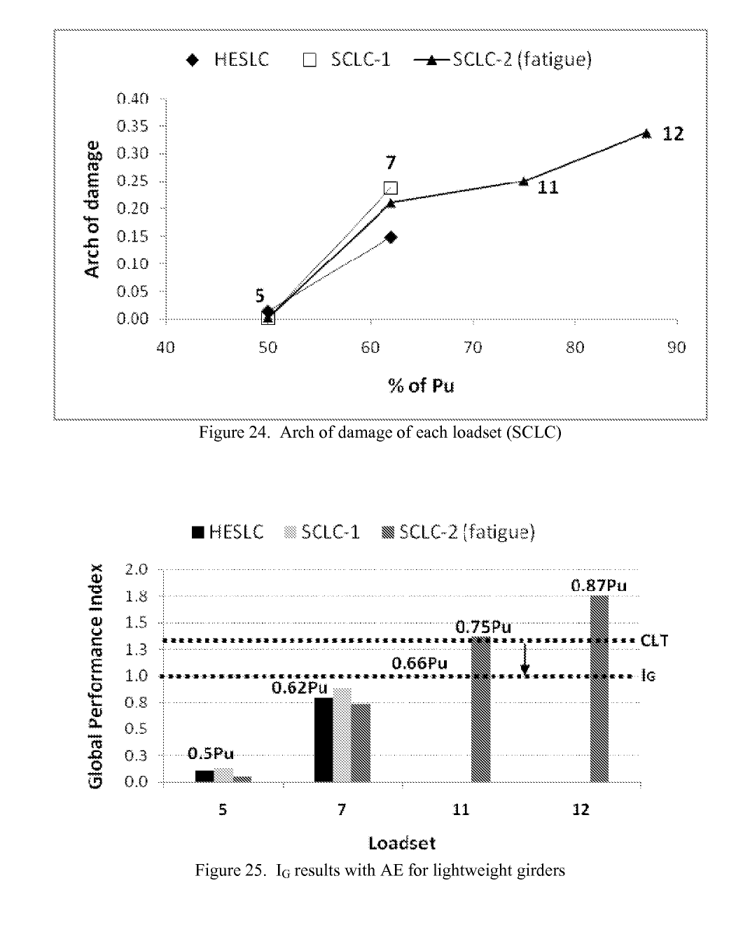

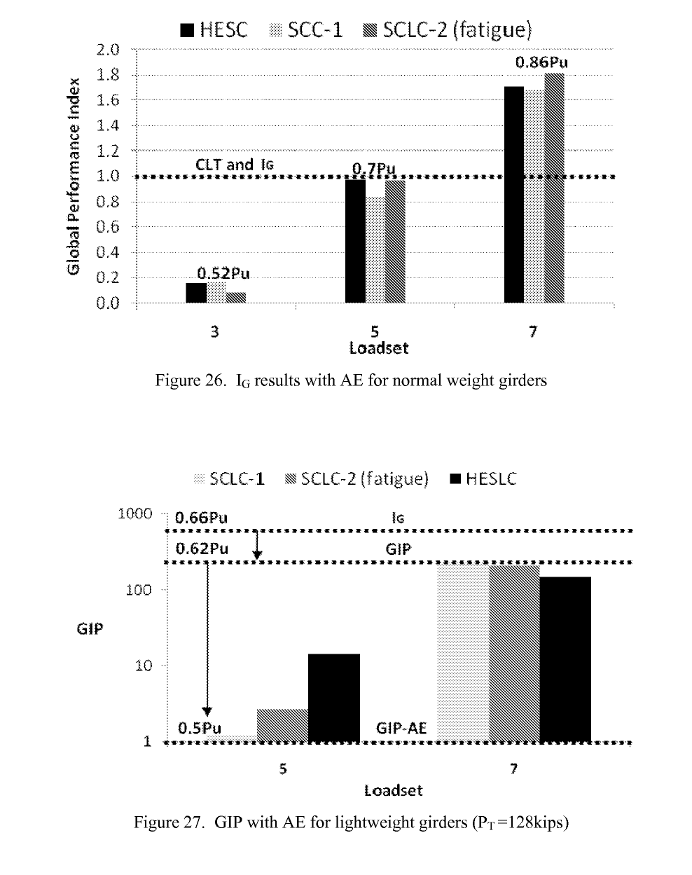

From FIGS. 25 and 26, the higher sensitivity of the global performance index is evident when the AE parameters are included in the calculations.

The addition of the I_{CRLR} in the computation of the I_{G} reduces the load required for the I_{G} to reach the unity from 75% to 66% for the lightweight girders, However, it is important to mention that the I_{G} parameters are tailored to indicate heavy damage in the structure and hence an integrity assessment of the members within the minor and intermediate levels of damage cannot be performed straightforwardly from I_{G} values.

In the case of the normal weight girders, the addition of the I_{CRLR} parameter did not decrease the level of load at which the criterion indicates significant deterioration. This situation is likely caused by the calibration of the I_{CRLR} for detection of damage within and above the intermediate damage level.

In order to exploit the high sensitivity of AE monitoring for damage diagnosis in prestressed flexural members, AE criteria should be tailored for damage detection at load levels within the minor damage region. Indication of deterioration inside the intermediate and heavy levels can be properly performed by the use of the GIP index previously presented.

In order to take full advantage of the high sensitive of AE for damage detection along with the objective delimitation of the damage thresholds by the GIP, a complimentary version of the GIP for damage detection within the minor-intermediate zone is proposed below. This formulation will only rely on the arch of damage (A_{D}), since cracking within the minor damage zone has practically no effect on the linear behavior of the member.

In order to attain this goal, an alternative definition of the GIP that corresponds to load levels within the minor-intermediate damage threshold based on the arch of damage is defined as:

GIP=Aₙβ⁻¹≦1.0 (10)

Where for lightweight,

$$\begin{matrix}β = 0.001 + 0.2 ( \frac{{P}_{T} - {P}_{O}}{{p}_{mt} - {P}_{O}} ) \\ And & ( 11 ) \\ {P}_{O} = {P}_{CR} + 0.1 ( {P}_{mt} - {P}_{CR} ) & ( 12 )\end{matrix}$$

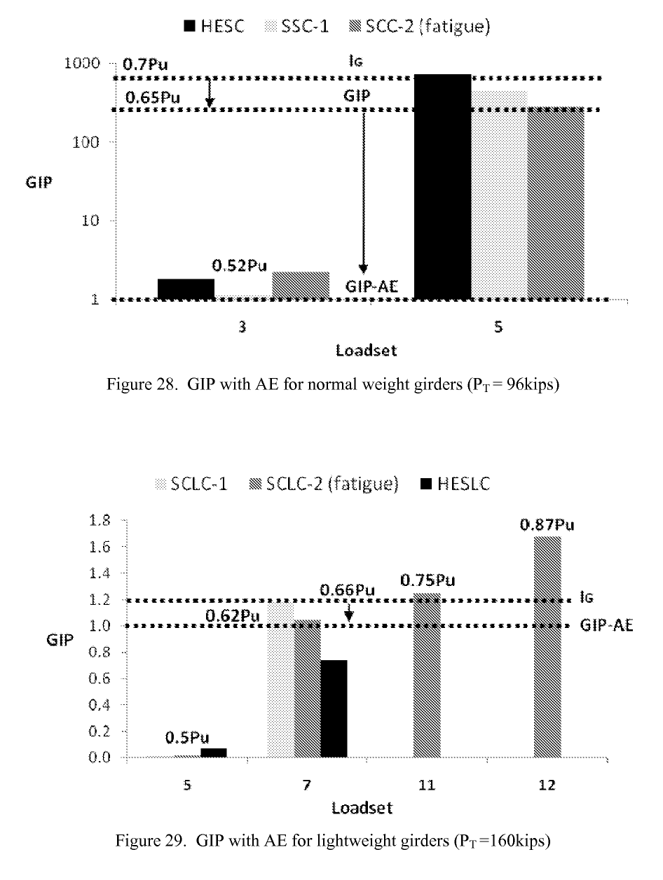

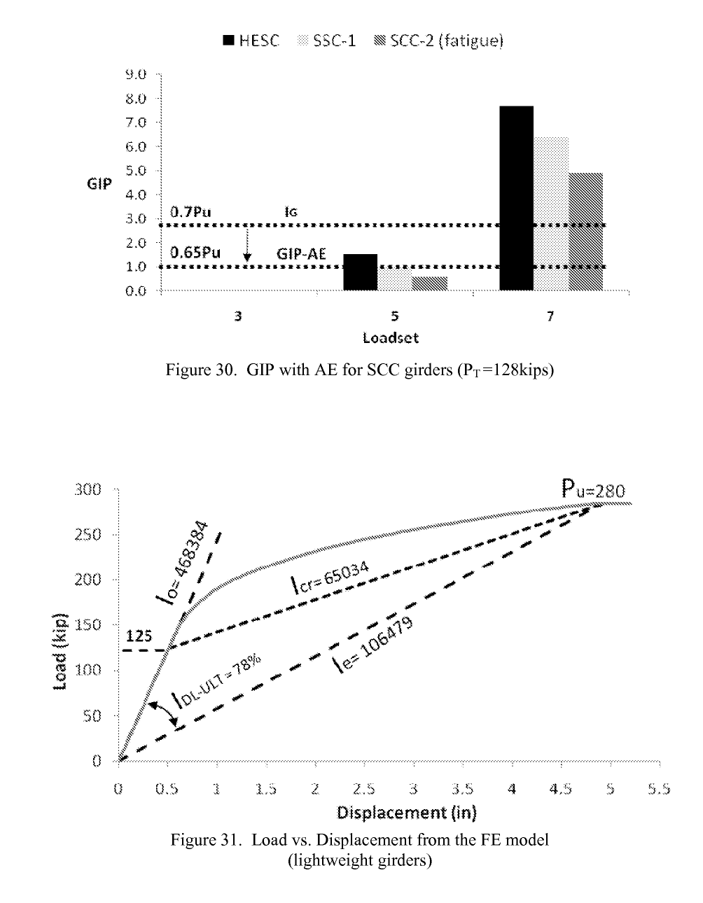

While for normal weight,

$$\begin{matrix}β = 0.0001 + 0.035 ( \frac{{P}_{T} - {P}_{O}}{{P}_{mt} - {P}_{O}} ) \\ And & ( 13 ) \\ {P}_{O} = {P}_{CR} + 0.31 ( {P}_{mt} - {P}_{CR} ) & ( 14 )\end{matrix}$$

Where A_{D} is the arch of damage from the CR vs. LR plot for any loadset, P_{T} is the target load at which the damage criterion should reach unity and which must be greater than Po, P_{CR} is the cracking load, and Pₘᵢ is the load at the theoretical minor-intermediate threshold. Computed GIP values are shown for SCLC specimens in FIG. 27 with a P_{T} equal to the peak load at loadset 5 (128 kips or 0.5 Pu), and in FIG. 29 with a P_{T} equal to the peak load at loadset 7 (160 kips or 0.62 Pu). In the case of the SCC specimens, GIP values are presented in FIG. 28 for a P_{T} of 96 kips (0.52 Pu) and in FIG. 30 for a P_{T} of 128 kips (0.65 Pu), corresponding to loadsets 3 and 5 respectively.

Results for both sets of girder specimens confirm that the GIP criterion using the A_{D} parameter provides the highest sensitivity among all the criteria previously presented (CLT, and I_{G}), reducing the load required for damage detection from 62% to 50% of Pu in the SCLC specimens, and from 65% to 52% of Pu in the SCC girders (FIGS. 27 and 28). This greater damage detection capability will result in a significant reduction in the load required for indication of damage during testing, to levels comparable to the cracking load of the girder considered as the point where deterioration begins.

TABLE 5
GIP with AE for lightweight girders

| | Loadset | 5 | 7 | 11 | 12 | | Pmi |
|---|---|---|---|---|---|---|---|
| Gider | % of Pu | 50 | 62 | 75 | 87 | Po (kip) | (kip) |
| HESLC | A_{D} | 0.014 | 0.1 | — | — | 128 | 160 |
| | GIP | 14 | 0.7 | — | — | | |
| SCLC-1 | A_{D} | 0.001 | 0.2 | | | | |
| | GIP | 1.2 | 1.2 | — | — | | |
| SCLC-2 | A_{D} | 0.003 | 0.2 | 0.3 | 0.3 | | |
| | GIP | 2.7 | 1.0 | 1.2 | 1.7 | | |

Pmi: Load at the minor-intermediate threshold

In addition, the AE version of the GIP offers the possibility to target any level of load within the Po and the load at the minor intermediate threshold. This feature allows triggering the detection of deterioration over 90% of the minor damage region for SCLC girders and within 70% of the same zone for SCC girders. In order to validate this capability, GIP values were computed targeting the load at loadset 7 (located right over the minor-intermediate limit) for SCLC girders and at loadset 5 (within the intermediate damage zone) for SCC specimens. Results from these calculations are presented in FIGS. 29 and 30 for both sets of girders.

TABLE 6
GIP with AE for normal weight girders

| | Loadset | 3 | 5 | 7 | | Pmin |
|---|---|---|---|---|---|---|
| Gider | % of Pu | 52 | 70 | 86 | Po (kip) | (kip) |
| HESC | A_{D} | 0.0002 | 0.07 | 0.4 | 96 | 120 |
| | GIP | 1.8 | 1.6 | 7.7 | | |
| SCC-1 | A_{D} | 0.0001 | 0.04 | 0.3 | | |
| | GIP | 1.2 | 1.0 | 6.4 | | |
| SCC-2 | A_{D} | 0.00022 | 0.03 | 0.2 | | |
| | GIP | 2.2 | 0.6 | 4.9 | | |

From FIGS. 29 and 30, it can be observed that the GIP performs satisfactorily in detecting deterioration within the intermediate damage region, considering that the criterion was specially tailored for damage detection at very low levels of deterioration. In the case of the SCLC girders, only the HESLC girder did not fail the criterion, while for the SCC specimens the beam SCC-2 was the only one that passed the test.

With all the results presented herein, it can be stated that the GIP in its two formulations constitutes an important advancement for integrity assessment of prestressed flexural members through load testing.

This novel formulation allows the diagnosis of damage according to a consistent methodology that takes into account the mechanical properties of the member and locates the damage thresholds accordingly, thus greatly reducing the subjectivity of the current evaluation criteria mostly based on the judgment of expert individuals.

In addition the proposed methodology separates the criteria for evaluation according to the targeted damage level, utilizing the deviation from linearity index (I_{DL}) for the intermediate and heavy damage zones where it can provide plentiful of information about the structural performance of the member, and applying the AE monitoring within the minor damage zone where its high sensitivity allows for the evaluation of members where cracking constitutes a critical variable that can hinder the structural performance as in cases of structures located in aggressive environments or members in special architectural applications.

The present disclosure describes the implementation of self-compacting lightweight concrete (SCLC) for the fabrication of prestressed bridge girders. In addition, a new approach for the objective identification of the damage zones in prestressed girders is described, taking into account the initial stiffness, the cracking moment, the ultimate capacity, and the fully cracked inertia of the member, so that a meaningful and coherent integrity assessment methodology can be formulated

In the case of the CLT, the global integrity parameter (GIP) based on the deviation from linearity index (I_{DL}), allows an improved integrity evaluation within the intermediate and heavy damage zones, by means of theoretical deterioration thresholds computed with specific mechanical features of the member under study.

Additionally, the arch of damage is proposed as a new AE criterion that can be incorporated in the GIP for damage detection at significant lower levels than the global performance index, and the CLT criteria evaluated independently.

Also a maximum test load corresponding to the minor-intermediate damage threshold is recommended for the structural evaluation of prestressed concrete girders where some damage is permitted during load testing. In all other cases, a target load within the minor damage zone can be used with the GIP based on the A_{D} parameter.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed:

1. A method for the estimation of damage zones in prestressed girders comprising:
selecting a prestressed girder for identification of damage zones; and
estimating damage zones by taking account cracking moment of the girder, ultimate load of the girder, fully cracked inertia of the girder, and elastic stiffness of the girder.

2. A method as in claim 1, wherein the cracking moment is defined as:
$${M}_{Cr} = {S}_{b} [ \frac{{P}_{O}}{{A}_{C}} ( 1 + \frac{e \times {C}_{b}}{{r}^{2}} ) + 7.5 λ \sqrt{{f}^{{′}_{c}}} ]$$
where Sb is the modulus of the composite section at the bottom fibers of the girder, Cb is the distance from the center of gravity of the girder section to the extreme tension fibers of the girder, Pe is the effective prestress force, Ac is the gross sectional area of the girder, e is the eccentricity of the tendons of the girder from the girder section center of gravity, r is the radius of gyration of the girder, and λ is equal to 1.0 for normal weight and 0.75 for lightweight concrete.

3. A method as in claim 1, wherein fully cracked inertia is defined as:
$${I}_{Cr} = {n}_{p} {A}_{p s} {d}_{p} ( 1 - 1.6 \sqrt{{n}_{p} \times {ρ}_{p}} )$$
where nₚ is the young modulus ratio, Aₚₛ is the area of prestressing steel, dₚ is the distance from the top of the section to the centroid of prestress, and ρₚ is the prestress reinforcing ratio.

4. A method as in claim 1, further comprising taking into account deviation from linearity at ultimate.

5. A method as in claim 4, wherein deviation from linearity at ultimate is defined as:
$${I}_{DLU} = ( 1 - \frac{{I}_{e}}{{I}_{O}} ) \times {10}_{0}$$

6. A method as in claim 5, further comprising estimating damage zones based, in part, on the following thresholds:
damage zones can be estimated as follows,
I_{DL-MINOR}≦0.2×I_{DLU}
0.2×I_{DLU}<I_{DL-INTERMEDIATE}≦0.45×I_{DLU}
0.45×I_{DLU}<I_{DL-HEAVY}

7. A method as in claim 1, wherein the girder is formed from self-consolidating lightweight concrete.

8. A method as in claim 1, wherein the girder is formed from self-consolidating concrete.

9. A method as in claim 1, wherein the girder is formed from high-early-strength concrete.

10. A method for the estimation of damage zones in prestressed girders comprising estimating damage zones by using a global integrity parameter (GIP).

11. A method as in claim 10, wherein GIP is defined as:
$$G I P = ( \frac{{I}_{DL}}{0.2 {I}_{DLU}} ) \leq 1.0$$
where I_{DLU} is the theoretical deviation from linearity at ultimate and the I_{DL} is the experimental deviation from linearity experienced by the girder at any load level during testing.

12. A method as in claim 10, wherein GIP is defined as:
GIP=Aₙβ⁻¹≦1.0
where for lightweight,
$$β = 0.001 + 0.2 ( \frac{{P}_{T} - {P}_{O}}{{P}_{mt} - {P}_{O}} )$$
$$and$$
$${P}_{O} = {P}_{CR} + 0.1 ( {P}_{mt} - {P}_{CR} )$$
while for normal weight,
$$β = 0.0001 + 0.035 ( \frac{{P}_{T} - {P}_{O}}{{P}_{mt} - {P}_{O}} )$$
$$and$$
$${P}_{O} = {P}_{CR} + 0.31 ( {P}_{mt} - {P}_{CR} )$$
and further where A_{D} is the arch of damage from the plot for any loadset, P_{T} is the target load at which the damage criterion should reach unity and which must be greater than Po, P_{CR} is the cracking load, and Pₘᵢ is the load at the theoretical minor-intermediate threshold.

13. A method as in claim 10, wherein the girder is formed from self-consolidating lightweight concrete.

14. A method as in claim 10, wherein the girder is formed from self-consolidating concrete.

15. A method as in claim 10, wherein the girder is formed from high-early-strength concrete.
